(19) United States
(12) Patent Application Publication
(10) Pub. No.: US 2009/0177119 A1
(43) Pub. Date: Jul. 9, 2009

(54) ARTICULATING INTRACORPOREAL MEDICAL DEVICE

(75) Inventors: MATT HEIDNER, MAPLE GROVE, MN (US); RICHARD C. GUNDERSON, MAPLE GROVE, MN (US); TED W. LAYMAN, PARK CITY, UT (US); ANDRZEJ MALEWICZ, MINNEAPOLIS, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., MAPLE GROVE, MN (US)

(21) Appl. No.: 11/969,210

(22) Filed: Jan. 3, 2008

(51) Int. Cl. A61M 25/09

(52) U.S. Cl. 600/585

(57) ABSTRACT

Guidewires and design, material, manufacturing method, and use alternatives for the same. An example method for advancing a guidewire to a target site in the anatomy of a patient may include providing the guidewire and advancing the guidewire through a blood vessel to a position adjacent the target site. Advancing the guidewire through a blood vessel may include disposing the hinge portion across a bifurcation in the blood vessel. The guidewire may have a proximal end and a distal end. The guidewire may include an elongate tubular member including a proximal portion, a distal portion extending to the distal end of the guidewire, and a hinge portion disposed between the proximal portion and the distal portion. The proximal portion may have a plurality of slots formed therein and a first lateral flexibility. The distal portion may have a plurality of slots formed therein and a second lateral flexibility. The hinge portion may have plurality of slots formed therein and a third lateral flexibility that is greater than both the first lateral flexibility and the second lateral flexibility.

### FIELD OF THE INVENTION

The invention relates to intracorporal medical devices, for example, intravascular medical devices. More particularly, the invention relates to intracorporal medical devices that include with a hinge portion or hinge member.

### BACKGROUND

A wide variety of intracorporal medical devices have been developed for medical use, for example, intravascular use. Some of these devices include guidewires, catheters, and other such devices. Of the known intracorporal medical devices that have defined flexibility characteristics, each has certain advantages and disadvantages. There is an ongoing need to provide alternative designs and methods of making and using medical devices.

### BRIEF SUMMARY

The invention provides design, material, manufacturing method, and use alternatives for medical devices. An example method for advancing a guidewire to a target site in the anatomy of a patient may include providing the guidewire and advancing the guidewire through a blood vessel to a position adjacent the target site. Advancing the guidewire through a blood vessel may include disposing the hinge portion across a bifurcation in the blood vessel. The guidewire may have a proximal end and a distal end. The guidewire may include an elongate tubular member including a proximal portion, a distal portion extending to the distal end of the guidewire, and a hinge portion disposed between the proximal portion and the distal portion. The proximal portion may have a plurality of slots formed therein and a first lateral flexibility. The distal portion may have a plurality of slots formed therein and a second lateral flexibility. The hinge portion may have plurality of slots formed therein and a third lateral flexibility that is greater than both the first lateral flexibility and the second lateral flexibility.

Another example method for advancing a guidewire to a target site in the anatomy of a patient may include providing a guidewire and advancing the guidewire through a blood vessel to a position adjacent the target site. Advancing the guidewire through a blood vessel may include disposing the hinge portion across a bifurcation in the blood vessel. The guidewire may include a tubular member having a plurality of slots formed therein. The tubular member may have a proximal portion having slots disposed therein in a first density, a hinge portion having slots disposed therein in a second density different from the first density, and a distal portion having slots disposed therein in a third density. The distal portion of the tubular member may extend to a distal end of the guidewire.

An example hinged guidewire may include a tubular member having a plurality of slots formed therein. The tubular member may have a proximal portion having slots disposed therein in a first density, a hinge portion having slots disposed therein in a second density different from the first density, and a distal portion having slots disposed therein in a third density that is the same as the first density. The second density may include more slots per unit length than both the first density and the third density. The distal portion of the tubular member may extend to a distal end of the guidewire.

Another example hinged guidewire may include a tubular member having a plurality of slots formed therein. The tubular member may have a proximal portion having a plurality of slots formed therein, a hinge portion having a plurality of slots formed therein, and a distal portion having a plurality of slots formed therein. The slots in the hinge portion may be aligned on opposite sides of the tubular member such that the hinge portion has a greater amount of lateral flexibility in directions oriented toward the slots and a decreased amount of lateral flexibility in directions oriented orthogonal to the slots. The distal portion of the tubular member may extend to a distal end of the guidewire.

### BRIEF DESCRIPTION OF THE DRAWINGS

FIG. 1 is partial cross-sectional view of an example guidewire;

FIG. 2 is a partial cross-section view of another example guidewire;

FIG. 3 is a partial cross-section view of another example guidewire;

FIG. 4 is a partial cross-section view of another example guidewire;

FIG. 5 is a partial cross-section view of another example guidewire;

FIG. 6 is a partial cross-section view of another example guidewire;

FIG. 7 is a partial cross-section view of another example guidewire;

FIG. 8 is a plan view of an example guidewire disposed within a portion of the vasculature of a patient;

FIG. 9 is a plan view of an example guidewire disposed within another portion of the vasculature of a patient;

FIG. 10 is a perspective view of another example guidewire;

FIG. 11 is an end view of another example guidewire;

FIG. 12 is a partial cross-section view of the example guidewire shown in FIG. 11;

FIG. 13 is another partial cross-section view of the example guidewire shown in FIG. 11;

FIG. 14 is a side view of another example guidewire;

FIG. 15 is a side view of another example guidewire;

FIG. 16 is a partial cross-section view of another example guidewire;

FIG. 17 is a side view of an example core wire;

FIG. 18 is a side view of another example core wire;

FIG. 19 is a side view of another example core wire;

FIG. 20 is a partial cross-sectional side view of a portion of another example guidewire;

FIG. 20A is a side view of the example guidewire shown in FIG. 20 with an alternative tubular member;

FIG. 21 is a plan view of the example guidewire shown in FIG. 20 disposed within a portion of the vasculature of a patient;

FIG. 22 is a partial cross-sectional side view of a portion of another example guidewire; and

FIG. 23 is a partial cross-sectional side view of a portion of another example guidewire.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

Weight percent, percent by weight, wt %, wt-%, % by weight, and the like are synonyms that refer to the concentration of a substance as the weight of that substance divided by the weight of the composition and multiplied by 100.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention. For example, although discussed with specific reference to guidewires in the particular embodiments described herein, the invention may be applicable to a variety of medical devices that are adapted to be advanced into the anatomy of a patient through an opening or lumen. For example, the invention may be applicable to fixed wire devices, catheters (e.g. balloon, stent delivery, etc.) drive shafts for rotational devices such as atherectomy catheters and IVUS catheters, endoscopic devices, laproscopic devices, embolic protection devices, spinal or cranial navigational or therapeutic devices, and other such devices.

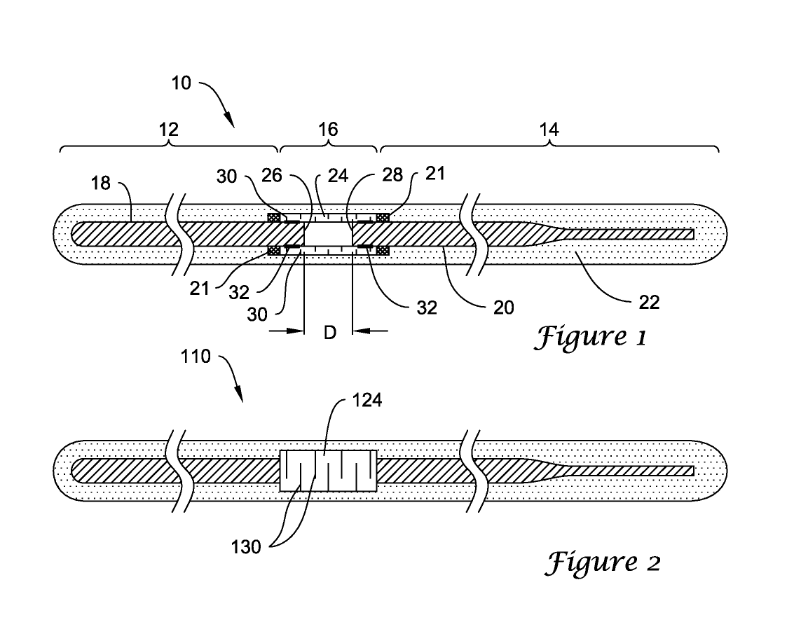

Refer now to FIG. 1, which is a partial cross-sectional view of an example guidewire 10. Guidewire 10 may include a proximal section 12, a distal section 14, and an articulating section 16. As used herein, the proximal section 12 and the distal section 14 may generically refer to any two adjacent guidewire sections along any portion of the guidewire. Those of skill in the art and others will recognize that the materials, structure, and dimensions of the proximal/distal guidewire sections 12/14 are dictated primary by the desired characteristics and function of the final guidewire, and that any of a broad range of materials, structures, and dimensions can be used.

The articulating section 16 is disposed at a location along the length of the guidewire 10 between proximal section 12 and distal section 14. Articulating section 16 may be adapted or configured to have flexibility characteristics that allow it to bend or flex to form relatively tight angles. Typically, the articulating section 16 has flexibility characteristics that make it more flexible than the adjacent portions of the proximal section 12 and distal section 14 of the guidewire 10. Articulating section 16 may also be configured or adapted for not only low force bending or flexing, but also for allowing torque and push forces to transfer from proximal section 12 to distal section 14. The articulating section 16 can be positioned at a location along the length of the guidewire such that when the device is used intracorporally at a particular location in the anatomy, the articulating section 16 corresponds with a particular part of the anatomy that requires the guidewire to bend or flex relatively aggressively during use. For example, in some embodiments, the articulating section is positioned at a location along the length of the device such that when the distal portion of the guidewire extends to a desired location within the anatomy of a patient, the articulating section 16 is disposed within a portion of the anatomy that requires the guidewire to make a relatively tight or aggressive bend or turn. Some of the other features and characteristics of articulating section 16 are described in more detail below.

The guidewire 10 can include one or more shaft or core portions. For example, the proximal section 12 of guidewire 10 may include a proximal shaft member 18. Similarly, distal section 14 may include a distal shaft member 20. The shaft members 18/20 may be distinct structures that can be connected or attached to one another and/or may be connected, but longitudinally spaced from each other, for example a distance D as shown in FIG. 1. Distance D can vary and may be in the range of about 10 centimeters or less. In some embodiments, the space defined by distance D may be left empty. Alternatively, the space may be filled with an appropriate material, for example, connector or binding material, radiopaque material, or the like. Alternatively, the central shaft or core portion can be one continuous member. For example, the proximal shaft member 18 and distal shaft member 20 may be continuous with one another and, collectively, define a continuous shaft or core. However, in such embodiments, the shaft or core portion includes a section within the articulating section 16 that includes increased flexability characteristics. Such increased flexability characteristics can be achieved through varying the material or structure of the shaft, as discussed in more detail below.

Shaft members 18/20 (in embodiments where shaft members 18/20 define a continuous core wire and in embodiments where shaft members 18/20 are distinct structures) may include metals, metal alloys, polymers, or the like, or combinations or mixtures thereof. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; combinations thereof, and the like; or any other suitable material.

As alluded to above, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2-0.44% strain before plastically deforming.

In some embodiments, the linear elastic and/or non-super-elastic nickel-titanium alloy is an alloy that does not show any martensite/austenite phase changes that are detectable by DSC and DMTA analysis over a large temperature range. For example, in some embodiments, there may be no martensite/austenite phase changes detectable by DSC and DMTA analysis in the range of about −60° C. to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties and has essentially no yield point.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of shaft members 18/20, or other structures included within guidewire 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of guidewire 10 in determining its location. Some examples of radiopaque materials can include, but are not limited to, gold, platinum, palladium, tantalum, tungsten alloy, polymer material loaded with a radiopaque filler, and the like. Additionally, radiopaque marker bands and/or coils may be incorporated into the design of guidewire 10 to achieve the same result.

In some embodiments, a degree of MRI compatibility is imparted into guidewire 10. For example, to enhance compatibility with Magnetic Resonance Imaging (MRI) machines, it may be desirable to make shaft members 18/20, or other portions of guidewire 10, in a manner that would impart a degree of MRI compatibility. For example, shaft members 18/20, or portions thereof, may be made of a material that does not substantially distort the image and create substantial artifacts (artifacts are gaps in the image). Certain ferromagnetic materials, for example, may not be suitable because they may create artifacts in an MRI image. Shaft members 18/20, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

As stated above, shaft members 18/20 can be made of the same material, or in some embodiments, can include portions or sections made of different materials. In some embodiments, the material used to construct guidewire 10 is chosen to impart varying flexibility and stiffness characteristics to different portions of guidewire 10. For example, proximal shaft member 18 and distal shaft member 20 may be formed of different materials, for example materials having different moduli of elasticity, resulting in a difference in flexibility. In some embodiments, the material used to construct proximal shaft member 18 can be relatively stiff for pushability and torqueability, and the material used to construct distal shaft member 20 can be relatively flexible by comparison for better lateral trackability and steerability. For example, proximal shaft member 18 can be formed of straightened 304v stainless steel wire or ribbon, and distal shaft member 20 can be formed of a straightened super elastic or linear elastic alloy, for example a nickel-titanium alloy wire or ribbon.

The length of shaft members 18/20 (and/or the length of guidewire 10) are typically dictated by the length and flexibility characteristics desired in the final medical device. For example, proximal section 12 may have a length in the range of about 20 to about 300 centimeters or more and distal section 14 may have a length in the range of about 3 to about 50 centimeters or more. It can be appreciated that alterations in the length of sections 12/14 can be made without departing from the spirit of the invention.

Shaft members 18/20 can have a solid cross-section, but in some embodiments, can have a hollow cross-section. In yet other embodiments, shaft members 18/20 can include combinations of areas having solid cross-sections and hollow cross sections. Moreover, shaft members 18/20 can be made of rounded wire, flattened ribbon, or other such structures having various cross-sectional geometries. The cross-sectional geometries along the length of shaft members 18/20 can also be constant or can vary. For example, FIG. 1 depicts shaft members 18/20 as having a round cross-sectional shape. It can be appreciated that other cross-sectional shapes or combinations of shapes may be utilized without departing from the spirit of the invention. For example, the cross-sectional shape of shaft members 18/20 may be oval, rectangular, square, polygonal, and the like, or any suitable shape.

As shown in FIG. 1, distal shaft member 20 may include one or more tapers or tapered regions. In some embodiments distal shaft member 20 may be tapered and have an initial outside size or diameter that can be substantially the same as the outside diameter of proximal shaft member 18, which then tapers to a reduced size or diameter. For example, in some embodiments, distal shaft member 20 can have an initial outside diameter that is in the range of about 0.010 to about 0.020 inches that tapers to a diameter in the range of about 0.001 to about 0.005 inches. The tapered regions may be linearly tapered, tapered in a curvilinear fashion, uniformly tapered, non-uniformly tapered, or tapered in a step-wise fashion. The angle of any such tapers can vary, depending upon the desired flexibility characteristics. The length of the taper may be selected to obtain a more (longer length) or less (shorter length) gradual transition in stiffness. Although FIG. 1 depicts distal shaft member 20 as being tapered, it can be appreciated that essentially any portion of guidewire 10 and/or shaft members 18/20 may be tapered and the taper can be in either the proximal or the distal direction. As shown in FIG. 1, the tapered region may include one or more portions where the outside diameter is narrowing, for example, the tapered portions, and portions where the outside diameter remains essentially constant, for example, constant diameter portions. The number, arrangement, size, and length of the narrowing and constant diameter portions can be varied to achieve the desired characteristics, such as flexibility and torque transmission characteristics. The narrowing and constant diameter portions as shown in FIG. 1 are not intended to be limiting, and alterations of this arrangement can be made without departing from the spirit of the invention.

The tapered and constant diameter portions of the tapered region may be formed by any one of a number of different techniques, for example, by centerless grinding methods, stamping methods, and the like. The centerless grinding technique may utilize an indexing system employing sensors (e.g., optical/reflective, magnetic) to avoid excessive grinding of the connection. In addition, the centerless grinding technique may utilize a CBN or diamond abrasive grinding wheel that is well shaped and dressed to avoid grabbing core wire during the grinding process. In some embodiments, distal shaft member 20 can be centerless ground using a Royal Master HI-AC centerless grinder.

As indicated above, the articulating section 16 is disposed at a location along the length of the guidewire 10 between proximal section 12 and distal section 14, and is adapted or configured to have flexibility characteristics that allows it to have an increased ability to bend or laterally flex to form relatively tight angles relative to the adjacent portions of the proximal section 12 and distal section 14. Typically, the articulating section 16 has flexibility characteristics that make it more laterally flexible than the adjacent portions of the proximal section 12 and distal section 14 of the guidewire 10. Those of skill in the art and others will recognize that the materials, structure, and dimensions of the articulating section 16 are dictated primary by the desired flexibility characteristics and function of the final guidewire, and that any of a broad range of materials, structures, and dimensions can be used.

In at least some embodiments, articulating section 16 may include or be defined by an articulating member 24. Articulating member 24 may be made from any appropriate structure and material including any of those described herein. In some embodiments, the articulating member 24 may be generally tubular so that it can couple a distal end 26 of proximal shaft member 18 and a proximal end 28 of distal shaft member 20. According to this embodiment, distal end 26 of proximal shaft member 18 and proximal end 28 of distal shaft member 20 may be disposed in opposite ends of the tubular articulating member 24. Ends 26/28 may be loosely disposed within articulating member 24 or ends 26/28 may be secured to articulating member 24. Securing may be achieved in a number of ways. For example, ends 26/28 may be secured to articulating member 24 by friction fitting, mechanically fitting, chemically bonding, thermally bonding, welding (e.g., resistance or laser welding), soldering, brazing, adhesive, the use of an outer sleeve or polymer layer to bond or connect the components, or the like, or combinations thereof. Some examples of suitable connection techniques are also disclosed in U.S. patent application Ser. Nos. 09/972,276, and 10/086,992, which are incorporated herein by reference. Additionally, in some embodiments, ends 26/28 may be secured to articulating member 24 by using an expandable alloy, for example a bismuth alloy. Some examples of methods, techniques and structures that can be used to interconnect different portions of a guidewire using such expandable materials are disclosed in a U.S. Patent Application entitled “Composite Medical Device” (Attorney docket number 1001.1546101) filed on even date with this application and which is hereby incorporated by reference.

FIG. 1 illustrates a plurality of bonding points 32, which may comprise any of the bonding or securing means described herein, disposed adjacent ends 26/28 and articulating member 24.

Lateral flexibility, bendability or other such characteristics of the articulating member 24 can be achieved or enhanced in a number of ways. For example, the materials selected for articulating member 24 may be chosen so that articulating section 16 has a greater lateral flexibility than the lateral flexibilities of proximal shaft member 18 adjacent distal end 26 and distal shaft member 20 adjacent proximal end 28. For example, articulating section 16 may be formed of materials having a different modulus of elasticity than the adjacent portions of the proximal shaft member 18 and distal shaft member 20, resulting in a difference in flexibility. Alternatively, articulating member 24 may include a thin wall tubular structure, made from essentially any appropriate material including those described herein, having desirable lateral flexibility characteristics.

In addition to, or as an alternative to material composition, the desired lateral flexibility or bending characteristics can be imparted or enhanced by the structure of the articulating member 26. For example, a plurality of grooves, cuts, slits, or slots 30 can be formed in a tubular articulating member 24. Such structure may be desirable because they may allow articulating member 24 to be bendable as well as transmit torque and pushing forces from proximal section 12 to distal section 14. The cuts or slots or grooves 30 can be formed in essentially any known way. For example, slots 30 can be formed by methods such as micro-machining, saw-cutting (e.g., using a diamond grit embedded semiconductor dicing blade), laser cutting, electron discharge machining, grinding, milling, casting, molding, chemically etching or treating, or other known methods, and the like. In some such embodiments, the structure of articulating member 24 is formed by cutting and/or removing portions of the tube to form slots 30. Some example embodiments of appropriate micromachining methods and other cutting methods, and structures for tubular and/or articulating members including slots and medical devices including tubular members are disclosed in U.S. Pat. Publication Nos. US 2003/0069522 and US 2004/0181174-A2; and U.S. Pat. Nos. 6,766,720; and 6,579,246, the entire disclosures of which are herein incorporated by reference. Some example embodiments of etching processes are described in U.S. Pat. No. 5,106,455, the entire disclosure of which is herein incorporated by reference. It should be noted that the methods for manufacturing guidewire 10 may include forming slots 30 in articulating member 24 using any of these or other manufacturing steps.

Various embodiments of arrangements and configurations of slots 30 are contemplated. In some embodiments, at least some, if not all of slots 30 are disposed at the same or a similar angle with respect to the longitudinal axis of the tubular and/or articulating member 24. As shown, slots 30 can be disposed at an angle that is perpendicular, or substantially perpendicular, and/or can be characterized as being disposed in a plane that is normal to the longitudinal axis of tubular and/or articulating member 24. However, in other embodiments, slots 30 can be disposed at an angle that is not perpendicular, and/or can be characterized as being disposed in a plane that is not normal to the longitudinal axis of tubular and/or articulating member 24. Additionally, a group of one or more slots 30 may be disposed at different angles relative to another group of one or more slots 30. The distribution and/or configuration of slots 30 can also include, to the extent applicable, any of those disclosed in U.S. Pat. Publication No. US 2004/0181174, the entire disclosure of which is herein incorporated by reference.

Slots 30 may be provided to enhance the flexibility of tubular and/or articulating member 24 while still allowing for suitable torque transmission characteristics. Slots 30 may be formed such that one or more rings and/or turns interconnected by one or more segments and/or beams are formed in tubular and/or articulating member 24, and such rings and beams may include portions of tubular and/or articulating member 24 that remain after slots 30 are formed in the body of tubular and/or articulating member 24. Such an interconnected ring structure may act to maintain a relatively high degree of torsional stiffness, while maintaining a desired level of lateral flexibility. In some embodiments, some adjacent slots 30 can be formed such that they include portions that overlap with each other about the circumference of tubular and/or articulating member 24. In other embodiments, some adjacent slots 30 can be disposed such that they do not necessarily overlap with each other, but are disposed in a pattern that provides the desired degree of lateral flexibility.

Additionally, slots 30 can be arranged along the length of, or about the circumference of, tubular and/or articulating member 24 to achieve desired properties. For example, adjacent slots 30, or groups of slots 30, can be arranged in a symmetrical pattern, such as being disposed essentially equally on opposite sides about the circumference of tubular and/or articulating member 24, or can be rotated by an angle relative to each other about the axis of tubular and/or articulating member 24. Additionally, adjacent slots 30, or groups of slots 30, may be equally spaced along the length of tubular and/or articulating member 24, or can be arranged in an increasing or decreasing density pattern, or can be arranged in a non-symmetric or irregular pattern. Other characteristics, such as slot size, slot shape and/or slot angle with respect to the longitudinal axis of tubular and/or articulating member 24, can also be varied along the length of tubular and/or articulating member 24 in order to vary the flexibility or other properties. In other embodiments, moreover, it is contemplated that the portions of the tubular member, such as a proximal section, a distal section, or the entire tubular and/or articulating member 24, may not include any such slots 30.

As suggested above, slots 30 may be formed in groups of two, three, four, five, or more slots 30, which may be located at substantially the same location along the axis of tubular and/or articulating member 24. Within the groups of slots 30, there may be included slots 30 that are equal in size (i.e., span the same circumferential distance around tubular and/or articulating member 24). In some of these as well as other embodiments, at least some slots 30 in a group are unequal in size (i.e., span a different circumferential distance around tubular and/or articulating member 24). Longitudinally adjacent groups of slots 30 may have the same or different configurations. For example, some embodiments of tubular and/or articulating member 24 include slots 30 that are equal in size in a first group and then unequally sized in an adjacent group. It can be appreciated that in groups that have two slots 30 that are equal in size, the beams (i.e., the portion of tubular and/or articulating member 24 remaining after slots 30 are formed therein) are aligned with the center of tubular and/or articulating member 24. Conversely, in groups that have two slots 30 that are unequal in size, the beams are offset from the center of tubular and/or articulating member 24. Some embodiments of tubular and/or articulating member 24 include only slots 30 that are aligned with the center of tubular and/or articulating member 24, only slots 30 that are offset from the center of tubular and/or articulating member 24, or slots 30 that are aligned with the center of tubular and/or articulating member 24 in a first group and offset from the center of tubular and/or articulating member 24 in another group. The amount of offset may vary depending on the depth (or length) of slots 30 and can include essentially any suitable distance.

Numerous other arrangements are contemplated that take advantage of the various arrangements and/or configurations discussed above.

In some embodiments, cuts or slots 30 can completely penetrate articulating member 24. In other embodiments, cuts or slots 30 may only partially extend into articulating member 24, or include combinations of both complete and partial cuts. In some embodiments, an elastic or low modulus filler material may be disposed within slots 30 to keep coating or sheath materials, such as the sheath 22, from filling in slots 30 and, possibly, reducing the flexibility of articulating member 24. Other embodiments lack such a filler.

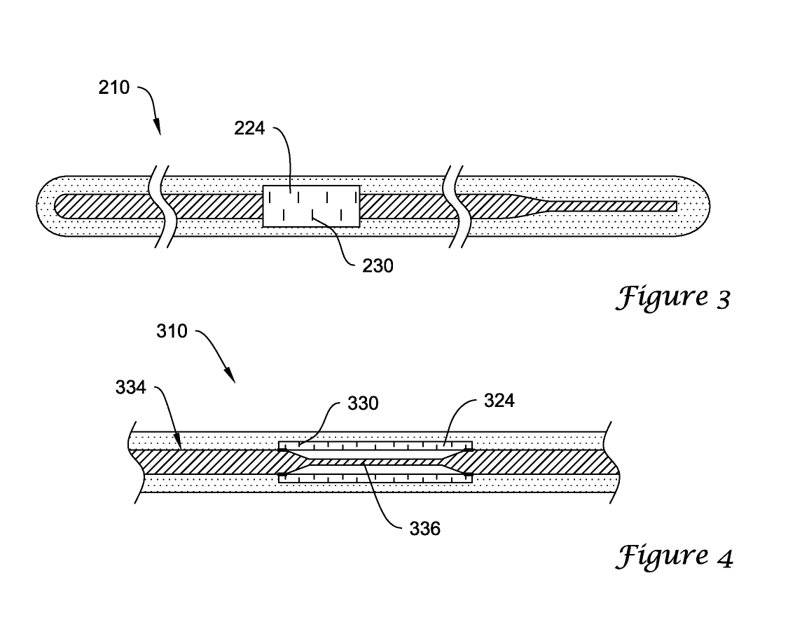

In some embodiments, some adjacent cuts or slots can be formed such that they include portions that overlap with each other about the circumference of the tube as suggested above. For example, FIG. 2 is a partial cross-sectional view of another example guidewire 110 that includes slots 130 disposed in an overlapping pattern. In other embodiments, some adjacent slots or cuts can be disposed such that they do not necessarily overlap with each other, but are disposed in a pattern that provides the desired degree of lateral flexibility. For example, FIG. 3 is a partial cross-sectional view of guidewire 210 that includes articulating member 224 including non-overlapping or opposing slots 230.

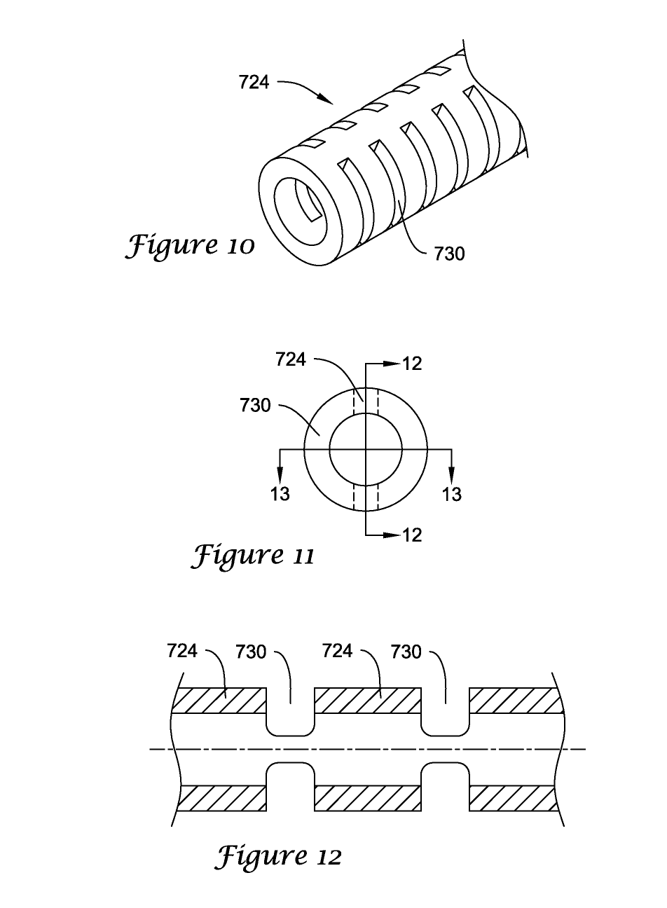

A number of additional variations in shape, arrangement, and pattern may be used. For example, another example articulating member 724, suitable for use with any of the devices described herein, is shown in FIG. 10. Articulating member 724 is similar to others described herein, except that slots 730 are rectangular in shape or pill-shaped, span nearly 180 degrees around articulating member 724, and are essentially disposed on opposite sides of articulating member 724. This figure illustrates a number of features of this and other articulating members. For example, the shape of slots 730 can vary to include essentially any appropriate shape. This may include having an elongated shape, rounded or squared edges, variability in width, and the like. Additionally, FIG. 10 illustrates that slots 730 may be arranged in a symmetrical pattern, such as being disposed essentially equally on opposite sides of articulating member 724, or in a non-symmetric or irregular pattern.

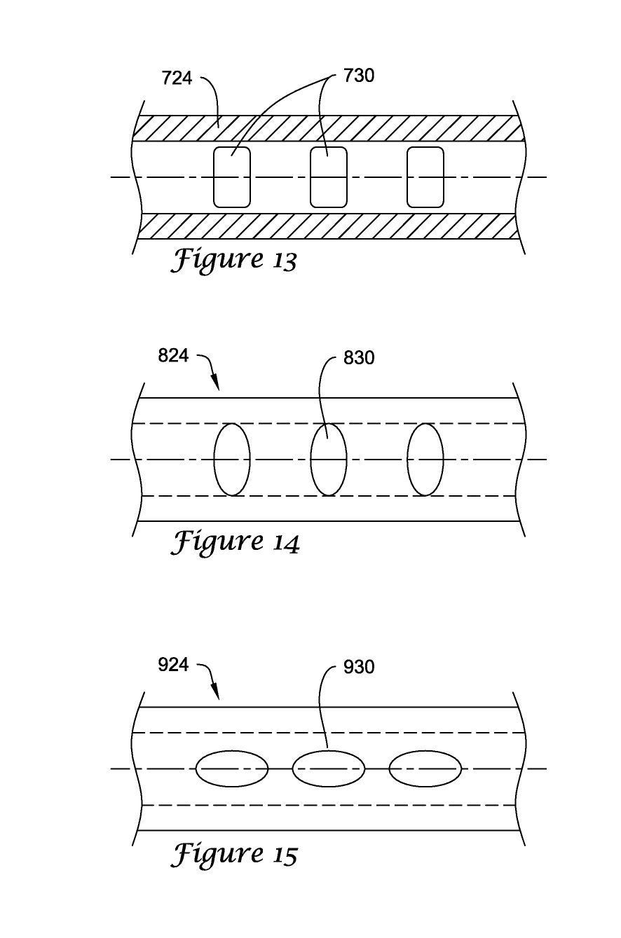

An end view of articulating member 724 is shown in FIG. 11. FIG. 11 shows the uncut areas of articulating member 724 (indicated by reference number 724) and the cut or slotted areas 730. Again, this figure illustrates that slots 730 may have a length that spans a significant portion of the circumference of articulating member 724, for example, approximating 180 degrees. For example, slots 730 may span about 175 degrees or less, 160 degrees or less, 145 degrees or less, 120 degrees or less, etc. The pattern of slots 730 can be observed by comparing FIG. 12 (which is a cross-sectional view taken through line 12-12 in FIG. 11) with FIG. 13 (which is a cross-sectional view taken through line 13-13 in FIG. 11).

Additionally, the size, shape, spacing, or orientation of the cuts or slots, or in some embodiments, the associated spines or beams, can be varied to achieve the desired lateral flexibility and/or torsional rigidity characteristics of the articulating member. Some examples of suitable shapes include squared, round, rectangular, oval, polygonal, irregular, and the like, or any other suitable shape. For example, FIG. 14 is a side of an example articulating member 824 having a plurality of oval slots 830. Similar to what is described above, the arrangement of slots 824 may vary. For example, FIG. 14 illustrates slots 830 arranged as a series of vertical ovals aligned side-by-side. Alternatively, FIG. 15 illustrates another example articulating member 924 with oval slots 930 arranged as a series of horizontal ovals aligned side-by-side. A number of addition arrangements may also be used. For example, the slots can be axially aligned, staggered, irregularly disposed, disposed either longitudinally or circumferentially (or both) about articulating member 824, or otherwise be in any suitable arrangement.

The number or density of the cuts or slots along the length of the articulating member may also vary, depending upon the desired characteristics. For example, the number or proximity of slots to one another near the midpoint of the length of the articulating member 24 may be high, while the number or proximity of slots to one another near either the distal or proximal end of the articulating member, or both, may be relatively low, or vice versa. Collectively, these figures and this description illustrate that changes in the arrangement, number, and configuration of slots may vary without departing from the scope of the invention. Some additional examples of arrangements of cuts or slots formed in a tubular body are disclosed in U.S. Pat. No. 6,428,489 and in Published U.S. patent application Ser. No. 09/746,738 (Pub. No. US 2002/0013540), both of which are incorporated herein by reference.

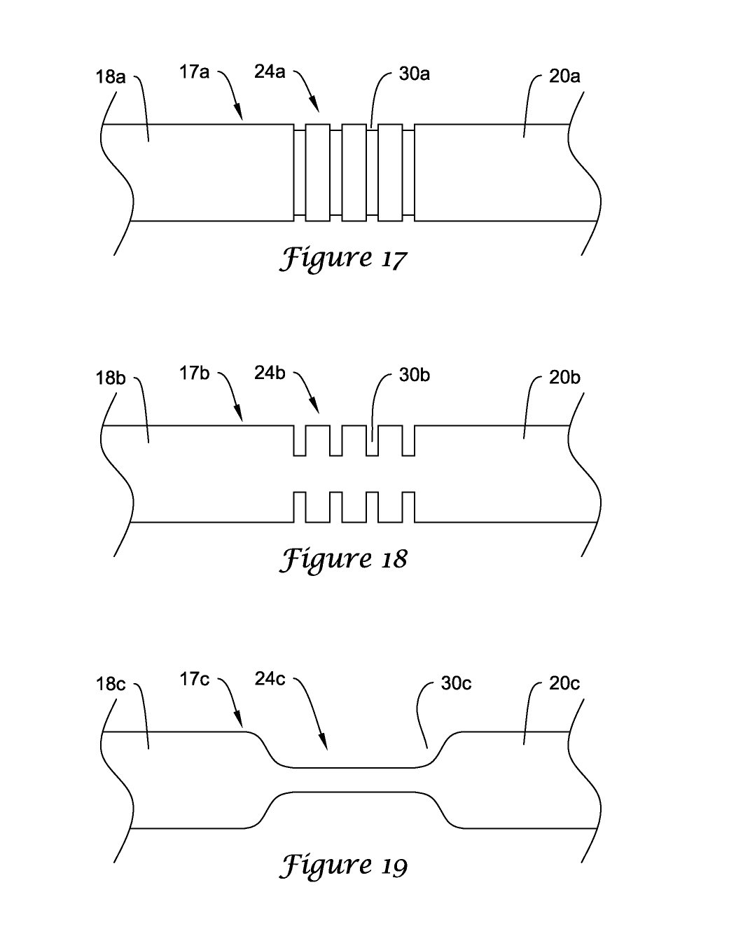

In other embodiments, the articulating section may include other structure to provide the desired increase in lateral flexibility. For example, the articulating section may include a hinge-like structure, for example a ball and socket type hinge, may include structural narrowing of all or portions of the guidewire shaft within the articulating region, may include cuts, slots, or grooves defined in the outer surface of the core wire or shaft, or other such structure. For example, FIG. 17 shows a plurality of grooves 30a formed in the outer surface of the core wire 17a at articulating section 24a. Similar to other core wires described herein, core wire 17a may include proximal section 18a and distal section 20a. Additionally, FIG. 18 shows a plurality of slots 30b formed in the outer surface of core wire 17b (including proximal section 18b and distal section 20b) at articulating section 24b. Moreover, FIG. 19 shows a necked-down or narrowing slot 30c defining articulating section 24c of core wire 17c (including proximal section 18c and distal section 20c).

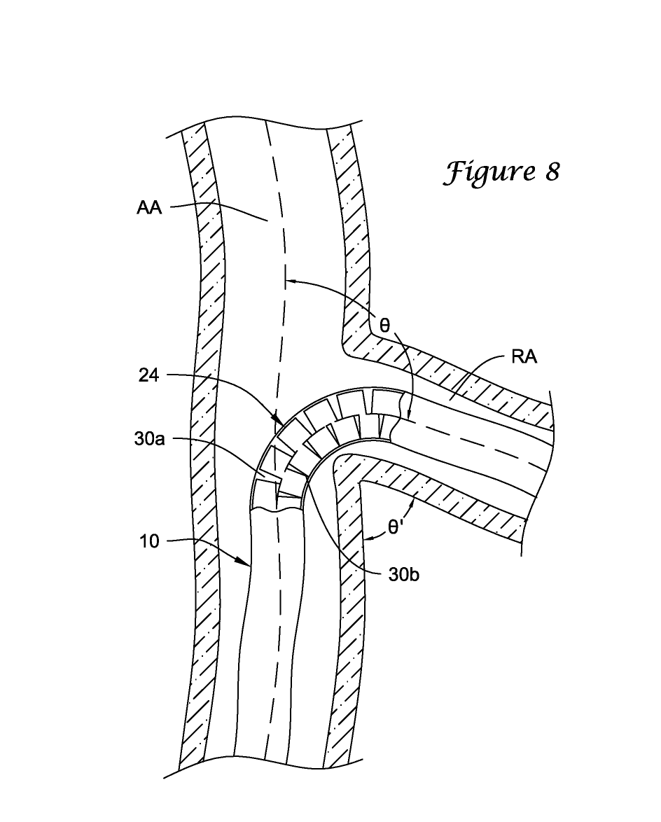

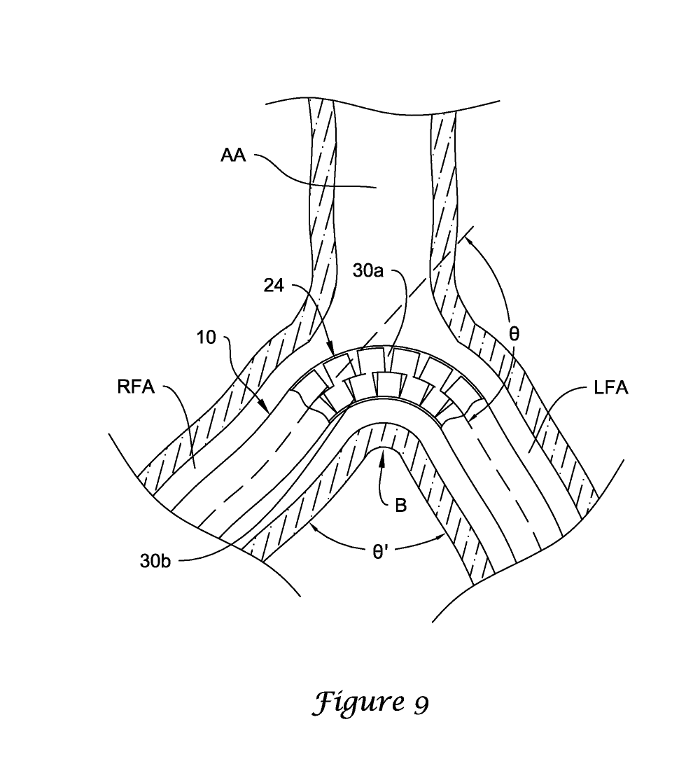

As stated above, the position of articulating section 16 can vary depending on the intended use of the guidewire 10. For example, uses of guidewire 10 may include navigating guidewire 10 across aggressive intravascular bends or curves in order to reach a target site or area. According to these embodiments, it may be desirable to position articulating section 16 so that it can correspond to these curves or bends when the distal region of the guidewire 10 is disposed adjacent the target site. For example, the vasculature may bend or curve such that guidewire 10 may need to bend 45 degrees or more, 60 degrees or more, 90 degrees or more, 120 degrees or more, etc. in order to navigate, span, or otherwise extend through the curve. As such, the articulating section 16 can be located at the appropriate position along the length of guidewire 10 so that articulating section 16 can be disposed within the bend when the distal guidewire section is located adjacent the target site. Articulating section 16, thus, enhances the ability of guidewire 10 to bend or laterally flex in accordance with the requirements of the anatomy being navigated. It should be noted that the above angles of guidewire 10 bending are understood to be angles that describe the change in course of the guidewire 10 and are shown in FIG. 8 as bending angle θ. As such, when the sharpness, tightness, and aggressiveness of the intravascular bend increases, the bending angle θ of the guidewire 10 increases.

Locating the articulating section 16 along the length of the guidewire in such a manner can be advantageous in maintaining the desired position of the guidewire, for example, the position of the distal portion of the guidewire relative to a target site. In at lease some conventional guidewire constructions that do not include an articulating section, the force necessary to bend the guidewire within an aggressive turn or bend in the anatomy results in a relatively high level of stress (i.e. tension and compressive forces) being produced in the guidewire shaft at the bending point. This stress can have adverse effects upon the ability of an operator to maintain the position of portions of the guidewire, for example, the distal tip at a desired location in the anatomy. For example, torsional rotation of the guidewire may cause the tip to move, or “whip” due to the stress. Additionally, the guidewire may have a greater tendency to slip or displace, for example, when the guidewire is rotated, or when catheter exchanges or other procedures are carried out that may place some additional force or movement on the guidewire. However, if an articulating section, as explained herein, is positioned along the length of the guidewire such that it is located within the aggressive turn or bend in the anatomy, the amount of stress can be reduced. As such, the desired positioning of the guidewire can be better maintained, for example, even during torsional rotation.

The particular distance of the location of the articulating member 24 from either the distal or proximal end of the guidewire can vary, depending upon, for example, the size or length of the anatomy of a patient, the particular location of the treatment site relative to the aggressive bend or turn in the anatomy, the lengths of the distal or proximal shaft members 18/20, and the like. Therefore, an entire series of devices is contemplated, each having one or more articulating members 24 being appropriately located along the length of the guidewire based upon the particular procedure being conducted and the particular anatomy of a patient.

One example of anatomy that can be navigated using a guidewire, but includes an aggressive bend or turn is the junction of the renal artery and the abdominal aorta in a human patient. The junction of the renal artery and the abdominal aorta may form a relatively aggressive angle, for example, an angle of about 90 degrees or more or less, when being approached from a femoral access point. A target site for treatment or navigation may be in a location adjacent to or within a renal artery or a kidney of a patient. Because of the angle formed in the anatomy at the junction of the renal artery and the abdominal aorta, it may be difficult for a distal portion of a medical device to maintain its position adjacent the target site while a portion of the wire must make the aggressive turn from the aorta to the renal artery. For example, in at lease some conventional guidewire constructions that do not include an articulating section, the force necessary to bend the guidewire within the turn in the anatomy may result in a relatively high level of residual stress in the guidewire shaft at the bending point. Thus, it may be desirable to use a guidewire including an articulating member 24 that is disposed at a location along the length of the guidewire such that when the distal portion of the guidewire is positioned at a desired location within or adjacent the target site, the articulating member 24 is positioned within the junction of the renal artery and the abdominal aorta. By including the articulating section 16 at such a location, the guidewire 10 can more easily access the renal artery when approached from a lower vascular region such as the femoral artery, and the amount of residual stress can be reduced.

In some such embodiments, the articulating section 16 can be disposed along the length of the guidewire at a location that is in the range of about 5 to about 25 centimeters from the distal end of guidewire 10. Of course the exact position can vary greatly as discussed above.

Another example of navigable anatomy that includes a relatively aggressive bend or turn is the aortic bifurcation at the base of the abdominal aorta. This is the point in the anatomy where the abdominal aorta splits and connects to the left and right femoral arteries. In some operations, it is desirable to gain access to one of the femoral arteries via a vascular access point in the other femoral artery. This requires that the guidewire (or other device) extends from one femoral artery to the other through the aortic bifurcation, which may form an angle of about 90 degrees or more or less when extending from one femoral artery to the other. Again, it may be desirable to use a guidewire including an articulating member 24 that is disposed at a location along the length of the guidewire such that when the distal portion of the guidewire is positioned at a desired location within or adjacent the target site, the articulating member 24 is positioned within the aortic bifurcation. By including the articulating section 16 at such a location, the guidewire 10 can more easily span the angle presented by the aortic bifurcation, and the desired positioning of the guidewire, for example the guidewire tip, can be better maintained. In some such embodiments, the articulating section 16 can be disposed along the length of the guidewire at a location that is in the range of about 20 to about 90 centimeters from the distal end of guidewire 10.

In some embodiments, the articulating member 24 may be generally described as being near the middle or the proximal end of guidewire 10. In other embodiments, the articulating member 24 may be generally described as being near the distal end of guidewire 10. Of course the exact position can vary greatly. According to these embodiments, guidewire 10 may include articulating member 24 disposed at other (including essentially any) position along guidewire 10.

FIG. 1 also illustrates that a coating or sheath 22 may be disposed over shaft members 18/20 and/or articulating member 16. In at least some embodiments, sheath 22 may be made from a polymer. However, any of the materials described herein may be appropriate. Some examples of suitable polymers may include polytetrafluroethylene (PTFE), fluorinated ethylene propylene (FEP), polyurethane, polypropylene (PP), polyvinylchloride (PVC), polyoxymethylene (POM), polybutylene terephthalate (PBT), polyphenylene sulfide (PPS), polyphenylene oxide (PPO), polysulfone, perfluroo (propyl vinyl ether) (PFA), polyether-ester (for example a polyether-ester elastomer such as ARNITEL® available from DSM Engineering Plastics), polyester (for example a polyester elastomer such as HYTREL® available from DuPont), polyamide (for example, DURETHAN® available from Bayer or CRISTAMID® available from Elf Atochem), elastomeric polyamides, block polyamide/ethers, polyether block ester, polyether block amide (PEBA, for example available under the trade name PEBAX®), silicones, polyethylene, Marlex high-density polyethylene, linear low density polyethylene (for example REXELL®), polyolefin, polyetheretherketone (PEEK), polyimide (PI), polyetherimide (PEI), nylon, other suitable materials, or mixtures, combinations, copolymers thereof, polymer/metal composites, lubricous polymers, and the like. In some embodiments sheath 22 can include a liquid crystal polymer (LCP) blended with other polymers to enhance torqueability. For example, the mixture can contain up to about 5% LCP. This has been found to enhance torqueability.

In some embodiments, sheath 22 is disposed over essentially the entire length of guidewire 10. This may include extending distally beyond distal shaft member 20. Sheath 22 may be disposed over shaft members 18/20 and/or articulating member 24 in any one of a number of different manners. For example, sheath 22 may be disposed by thermal bonding techniques, by coating, by extrusion, co-extrusion, interrupted layer co-extrusion (ILC), or fusing several segments end-to-end. The layer or layers may have a uniform stiffness or a gradual reduction in stiffniess from the proximal end to the distal end thereof. The gradual reduction in stiffness may be continuous as by ILC or may be stepped as by fusing together separate extruded tubular segments. Sheath 22 may be impregnated with a radiopaque filler material to facilitate radiographic visualization. Those skilled in the art will recognize that these materials can vary widely without deviating from the scope of the present invention.

In some embodiments, wherein the sheath 22 is disposed over the articulating section 16, it may be desirable that the sheath is disposed in such a manner that the structure within the articulating section 16, for example, an articulating member 24, can still flex or bend in an acceptable manner. For example, the portion of the sheath 22 that extends over the articulating section 16 can be made of a suitably flexible material. Additionally, in some embodiments, the sheath 22 may extend over the articulating member 24, but is not directly attached thereto, such that, for example, the slots or grooves in the articulating member can flex and move within the sheath as it flexes or bends.

In some embodiments, one or more second coating or sheath (not shown), for example a lubricious, a hydrophilic, a hydrophobic, a protective, or other type of coating may be applied over portions or all of sheath 22 and/or guidewire 10. Hydrophobic coatings such as fluoropolymers provide a dry lubricity which can improve guidewire handling and device exchanges. Lubricious coatings can also improve steerability and lesion crossing capability. Suitable lubricious polymers are well known in the art and may include silicone and the like, hydrophilic polymers such as polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference. It may be desirable to include a plurality of different second coating, for example having different properties or lubricities. For example, it may be desirable to include a more lubricous second coating the distal end of guidewire 10 and a less lubricious second coating (which may aid the ability of the clinician to grasp guidewire 10) near the proximal end of guidewire 10.

FIG. 4 illustrates another example guidewire 310. Guidewire 310 is similar to other guidewires described herein except that it shows an example configuration where proximal and distal shaft members define a core wire 334. Core wire 334 may include a narrowed or tapered articulating section 336 (generally disposed at an articulating section of guidewire 310 that is positioned similar to articulating section 16 of guidewire 10 in FIG. 1), disposed between continuous proximal and distal shaft members 318/320. Tapered articulating section 336 may be formed according to a number of different techniques such as grinding methods described herein and others. Similar to what is described above, articulating member 324 may include one or more cuts or slots 330.

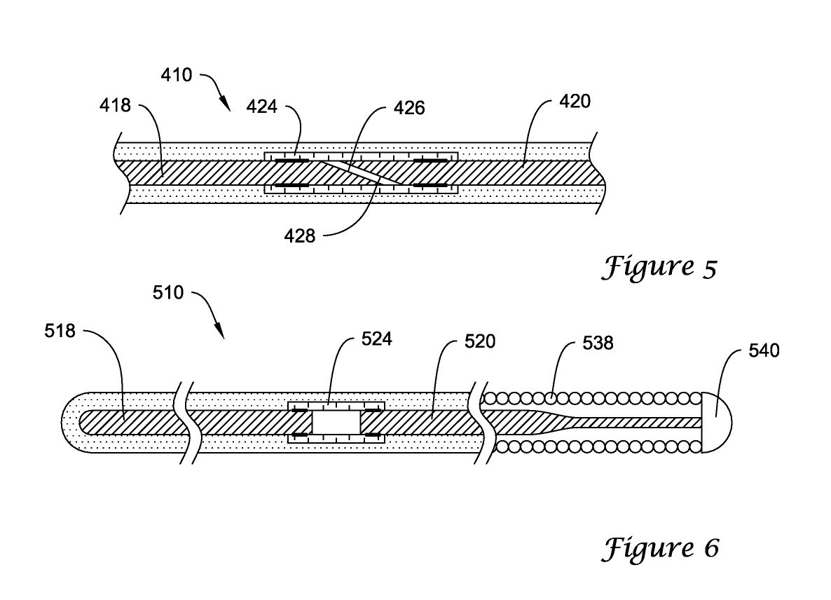

Another example guidewire 410 is shown in FIG. 5. Guidewire 410 is similar to other guidewires disclosed herein except that distal end 426 of proximal shaft member 418 and proximal end 428 of distal shaft member 420 may be angled. Articulating member 424 may be disposed over ends 426/428. In at least some embodiments, a portion of proximal and distal shaft members 418/420 may overlap. This may allow any transitions in flexibilities between shaft members 418/420 to be more gradual or smooth.

FIG. 6 is a partial cross-sectional view of another example guidewire 510. Guidewire 510 is similar to other guidewires described herein except that it includes a spring tip characterized by a distal coil 538 and a distal tip 540. Guidewire 510 may also include proximal shaft member 518, distal shaft member 520, and articulating member 524. It can be appreciated that a number of other types of guidewire tips (for example, shapeable tips, other atraumatic tips, and the like) are known in the art and may be used with any of the guidewire described herein without departing from the spirit of the invention.

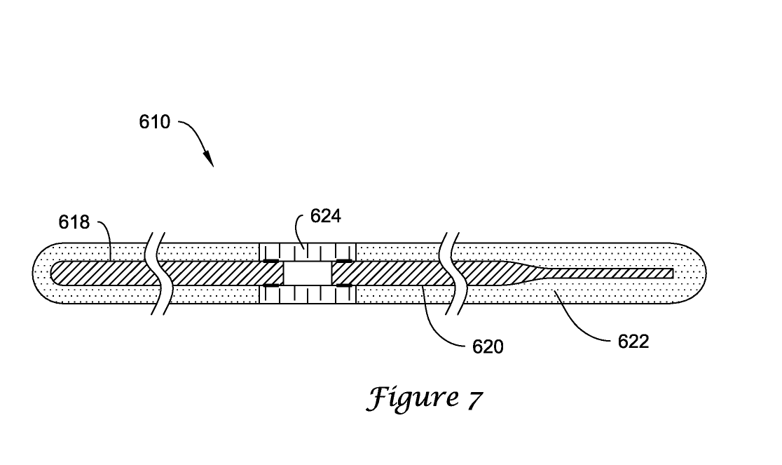

FIG. 7 is a partial cross-sectional view of another example guidewire 610. Guidewire 610 is similar to other guidewires described herein except that articulating member 624, coupling shaft members 618/620, and sheath 622 are aligned so that at least a portion of articulating member 624 is not covered by the sheath 622.

FIG. 8 illustrates an example plan view of the use of guidewire 10 (that is similarly applicable to any of the guidewires disclosed herein) with articulating member 24 spanning the transition between the abdominal aorta AA and the renal artery RA. The renal artery RA may be disposed at an angle θ′ relative to the abdominal aorta AA. In order to span the transition, the guidewire 10 may need to bend at an angle θ, which may be in the range of about 45 degrees or greater, 60 degrees or greater, 90 degrees or greater, 120 degrees or greater, etc. The features, characteristics, and benefits of guidewire 10 may be utilized at other intravascular locations including, for example, peripheral intravascular locations as well as cardiac locations. For example, it may be desirable to dispose articulating member 24 at branching point or fork where abdominal aorta AA splits to the left and right femoral arteries.

Because angle θ′, as it can be seen, may be about ninety degrees or more or less, articulating member 24 may act as a hinge or elbow that spans the relevant transition point that may, for example, allow guidewire 10 to better hold its position while still maintaining its ability to transmit torque and other forces. It can also be seen in FIG. 8 that slots 30 within articulating member 24 may be able to alter their position when bending across a transition point. For example, FIG. 8 illustrates that some of slots 30, indicated by reference number 30a, may be opened or widened while others, indicated by reference number 30b, may be closed or narrowed. The opening or narrowing of the slots indicate that the articulating member can be adapted or configured to compensate for the tensional and compressive forces that are being placed on the articulating member as it spans the bend or turn in the anatomy.

FIG. 9 similarly illustrates another example plan view of the use of guidewire 10, or any of the other guidewires described herein, with articulating member 24 is disposed adjacent the bifurcation B where the abdominal aorta AA splits into the left femoral artery LFA and the right femoral artery RFA. According to this embodiment, guidewire 10 can be used to access one femoral artery, for example the left femoral artery LFA, by advancing guidewire 10 from the right femoral artery RFA, across the bifurcation B in the abdominal aorta AA, and into the left femoral artery LFA. Similar to what is described above, right femoral artery RFA and left femoral artery LFA may be disposed at angle θ′ relative to each other, which may be about 90 degrees or less. Accordingly, guidewire 10 may need to bend at an angle θ, which may be in the range of about 45 degrees or greater, 60 degrees or greater, 90 degrees or greater, 120 degrees or greater, etc.

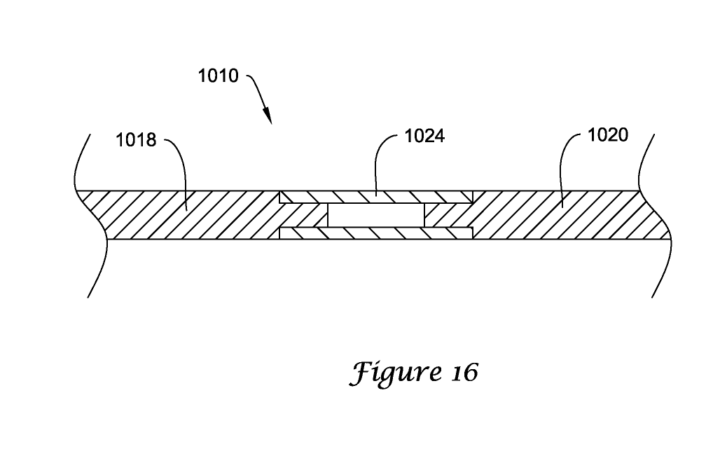

FIG. 16 illustrates another example guidewire 1010. Guidewire 1010 is similar to other guidewires described herein. For example, guidewire 1010 may include proximal shaft member 1018, distal shaft member 1020, and articulating member 1024. However, proximal and distal shaft members 1018/1020 may be stepped or necked down so that articulating member 1024 can be disposed over the ends thereof Accordingly, guidewire 1010 may have a smooth outer surface, defined by shaft members 1018/1020 and articulating member 1024, and may not need to include an outer sheath.

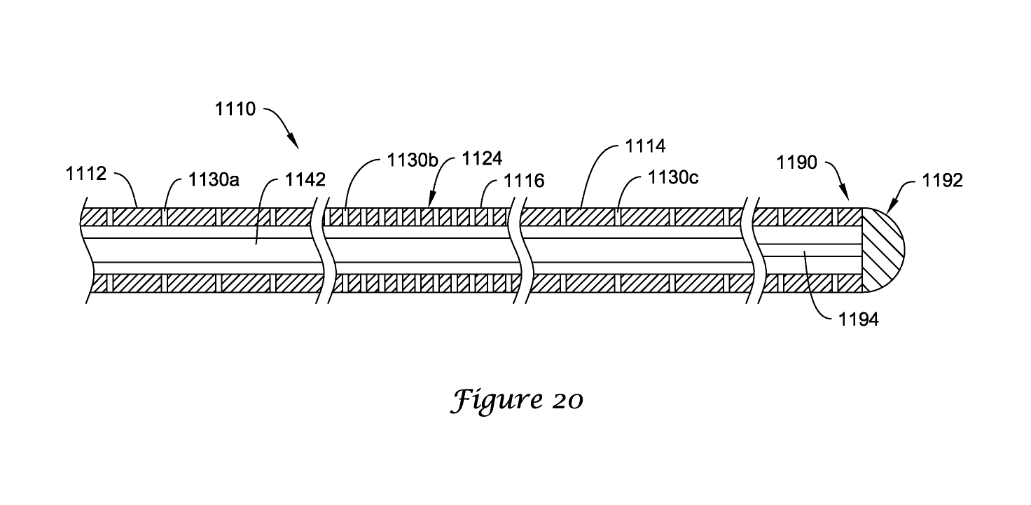

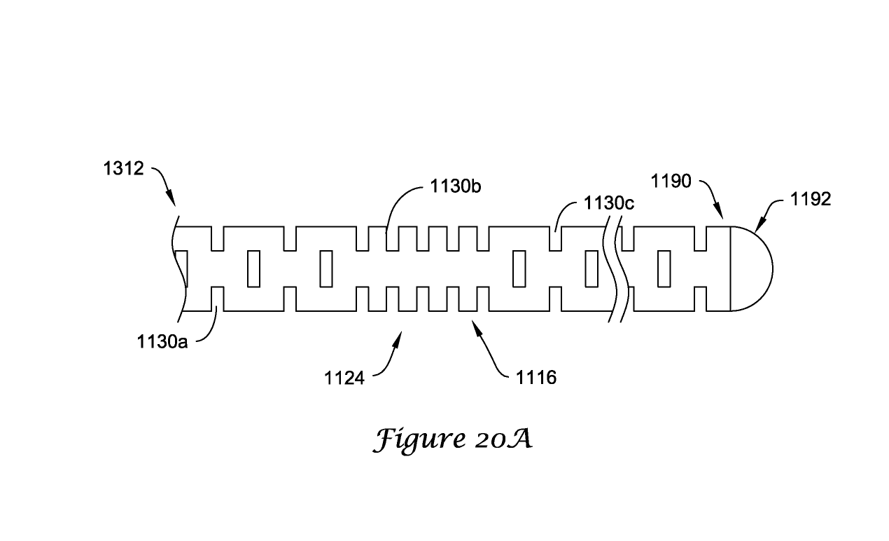

FIG. 20 illustrates another example guidewire 1110 that may be similar in form and function to any of the other guidewires disclosed herein. Guidewire 1110 may include an elongate tubular member 1124. In some embodiments, tubular member may extend a substantial portion of guidewire 1110 and/or a substantial portion of the distal section of guidewire 11 10. For example, tubular member 1124 may include a proximal portion 1112 and a distal portion 1114, and distal portion 1114 may extend to the distal end 1190 of guidewire 1110. Tubular member 1124 may also include a hinge member or hinge portion 1116 disposed between proximal portion 1112 and distal portion 11 14. In some embodiments, hinge portion 1116 may be similar to some or all of the articulating sections/members disclosed herein.

In some embodiments, tubular member 1124 may be disposed over a core or shaft member 1142. Shaft member 1142 may be similar to any of the shaft members or shaft member portions disclosed herein such as the various proximal shaft members and/or distal shaft member disclosed herein or any of the various combinations thereof. In some embodiments, shaft member 1142 may include an articulating member (not shown) that may be similar to any of the articulating members disclosed herein. In other embodiments, shaft member 1142 may be formed of a single monolith of material. In still other embodiments, shaft member 1142 may include two or more portions that attached together in any suitable way other than through the use of articulating member. In some embodiments, shaft member 1142 may extend to distal end 1190 of guidewire 1110 and/or distal portion 1114 of tubular member 1 124. Alternatively, tubular member 1124 may extend distally beyond the distal end of shaft member 1 142. In at least some of these embodiments, a shaping member 1194 may be attached to or otherwise coupled to shaft member 1142.

Tubular member 1124 may have a plurality of slots, for example slots 1130a/1130b/1130c, formed therein that may be similar to any of the other slots disclosed herein. Slots 1130a/1130b/1130c may provide guidewire 1110 with desirable flexibility and/or torque transmitting characteristics. In at least some embodiments, slots 1130a/1130b/1130c may be formed in proximal portion 1112, hinge portion 1116, and distal portion 1114, respectively.

In some embodiments, some of slots 1130a/1130b/1130c are arranged in different densities and/or configurations with a different number of slots per unit length. For example, slots 1130a may be disposed along proximal portion 1112 in a first density and slots 1130b may be disposed along hinge portion 1116 in a second density that is different from the first density. In some embodiments, the second density of slots 1130b may include more slots per unit length. This may provide hinge portion 1116 with an increased amount of lateral flexibility relative to proximal portion 1112.

Slots 1130c may be disposed along distal portion 1114 in a third density. In some embodiments, the third density includes a different number of slots per unit length than both the first density and the second density. This may provide distal portion 1114 with a lateral flexibility that is different from that of both proximal portion 1112 and hinge portion 1116. In other embodiments, the third density may be the same as the first density. This may result in hinge portion 1116 having a different (e.g., greater) lateral flexibility that its flanking portions 1112/1114. In still other embodiments, the third density may be the same as the second density.

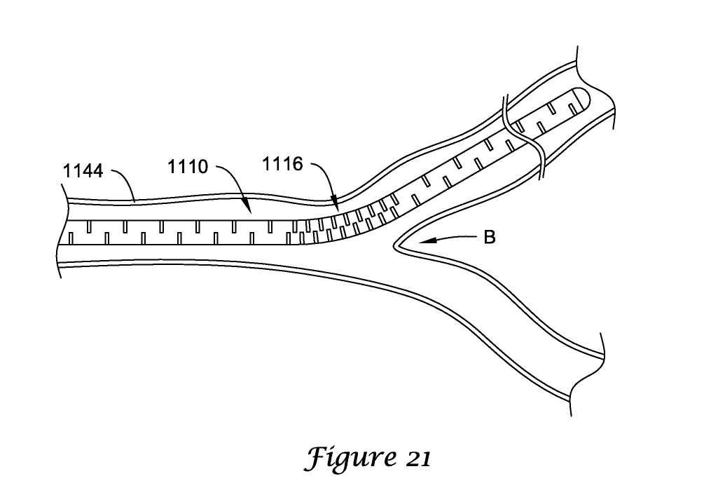

In at least some embodiments, hinge portion 1116 has a greater amount of lateral flexibility than both proximal portion 1112 and distal portion 1114. This arrangement may be desirable for a number of reasons. For example, by virtue of having hinge portion 1116 with an increased lateral flexibility relative to proximal and distal portions 1112/1114, guidewire 1110 may be able to more easily pass through a bifurcation B in a blood vessel 1144 when advancing guidewire 1110 through blood vessel 1144 toward a target site as shown in FIG. 21. This may be because hinge portion 1116 may more easily bend around or otherwise span the bifurcation B, for example, in a manner similar to how the articulating sections and/or members disclosed herein are able to span a bifurcation or otherwise transition between blood vessel in the vasculature. In at least some embodiments, advancing guidewire 1110 to an intravascular target site may include disposing hinge portion 1116 across bifurcation B. It can be appreciated that blood vessel 1114 may comprise portions of the vasculature described above including any of the vessels shown in FIGS. 8-9. As such, guidewire 1110 may be advanced through these portions of the vasculature, as well as others, and such advancing may include disposing hinge portion 1116 across a transition area or bifurcation B.

Hinge portion 1116 may include slots 1130b arranged in a number of different ways including any suitable arrangement disclosed herein. In some embodiments, slots 1130b are longitudinally aligned on opposite sides of tubular member 1124 as shown in FIG. 20A. This may allow hinge portion 1116 to have greater lateral flexibility in directions that are oriented toward the rows of slots 1130b. In directions orthogonal (i.e., 90 degrees) to the rows of slots 1130b, hinge portion 1116 may have a decreased lateral flexibility. In some embodiments, the amount of lateral flexibility in these orthogonal directions may be the same, greater, or less than the amount of lateral flexibility in proximal portion 1312, distal portion 1114, or both. Slots 1130b may be absent along other portions of hinge portion 1116 so that hinge portion has increased lateral flexibility only in the two directions that are pointed toward the rows of slots 1130b and less lateral flexibility in essentially all other directions. In other embodiments, hinge portion 1116 may have a different or more regular pattern so that it may have the same lateral flexibility in essentially all directions. Tubular members like that shown in FIG. 20A may be utilized in any of the guidewires disclosed herein.

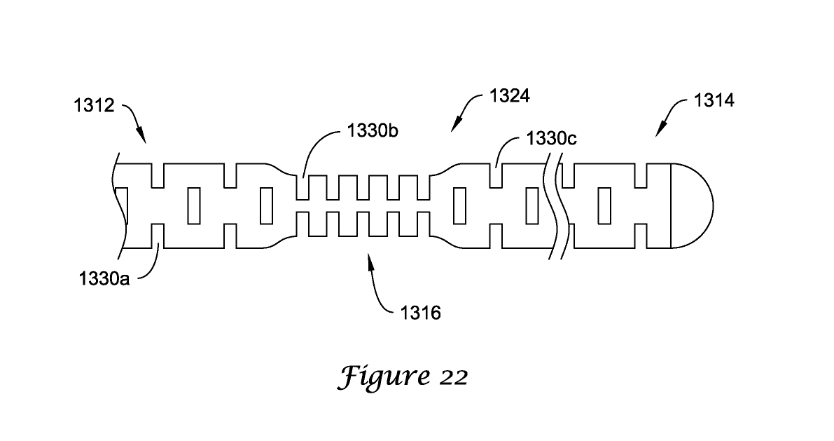

FIG. 22 illustrates another example that may be similar to form and function to any of the other guidewires disclosed herein. The guidewire illustrated in FIG. 22 may include a tubular member 1324 having a proximal portion 1312, a distal portion 1314, and a hinge portion 1316 disposed therebetween. Distal portion 1314 may extend to the distal end of the guidewire. Proximal portion 1314, hinge portion 1316, and distal portion 1314 may have a plurality of slots 1330a, 1330b, and 1330c, respectively, formed therein. In some embodiments, proximal portion 1312 has a first outer diameter, distal portion 1314 has a second outer diameter, and hinge portion 1316 has a third outer diameter that is smaller than the first outer diameter, the second outer diameter, or both.

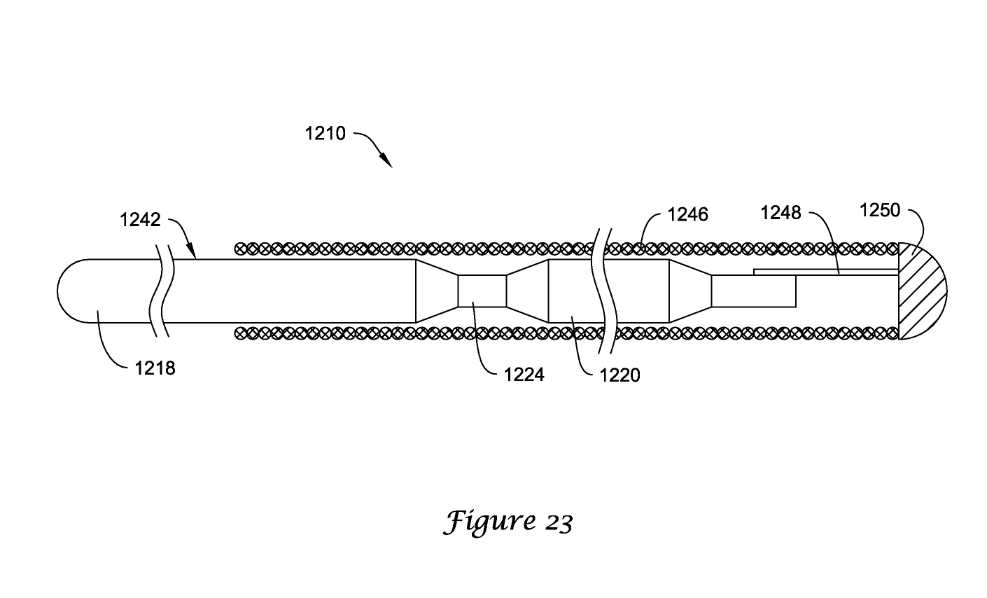

FIG. 23 illustrates another example guidewire 1210 that may be similar to form and function to any of the other guidewires disclosed herein. Guidewire 1210 may include a core or shaft member 1242 having a proximal portion 1218, a distal portion 1220, and a hinge portion 1224 disposed therebetween. In at least some embodiments, hinge portion 1224 may have a different outer diameter or outer perimeter than proximal portion 1218, distal portion 1220, or both. For example, hinge portion 1124 may have a smaller outer diameter than both proximal portion 1218 and distal portion 1220. This may provide a number of desirable features for guidewire 1210. For example, this arrangement may allow hinge portion 1224 to bend around a portion of the anatomy such as a bifurcation in a blood vessel much like any of the articulating portions/sections/members disclosed herein. As such, guidewire 1210 may be advanced through the vasculature in any of the manners described above. In some embodiments, hinge portion 1224 may only change in outer diameter. In other embodiments, hinge portion 1224 may also change in shape. For example, hinge portion 1224 may be flattened or otherwise change to resemble a ribbon.

Guidewire 1210 may include a number of additional structural components that may be common to guidewires. For example, guidewire 1210 may include a coil 1246 extending over a portion of shaft member 1242. In other embodiments, a tubular member (e.g., a tubular member having slots such as any suitable tubular members disclosed herein) may be used in place of or in addition to coil 1246. A shaping member 1248 may be coupled to shaft member 1242 (for example distal portion 1220 of shaft member 1242). Shaping member 1248 may be made from a relatively inelastic material so that a clinician can bend or shape the distal end of guidewire 1210 into a shape that may facilitate navigation of guidewire 1210 through the anatomy. A tip member 1250 may also be coupled to shaft member 1242 that may define an atraumatic distal tip of guidewire 1210. In general, tip member 1250 may include solder. However, other versions of tip member 1250 are contemplated including tip members 1250 that comprise or form a polymeric tip.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What is claimed is:

1. A method for advancing a guidewire to a target site in the anatomy of a patient, the method comprising:
providing the guidewire, the guidewire having a proximal end and a distal end, wherein the guidewire includes an elongate tubular member including:
a proximal portion having a plurality of slots formed therein and a first lateral flexibility,
a distal portion extending to the distal end of the guidewire, the distal portion having a plurality of slots formed therein and a second lateral flexibility, and
a hinge portion disposed between the proximal portion and the distal portion, the hinge portion having plurality of slots formed therein and a third lateral flexibility that is greater than both the first lateral flexibility and the second lateral flexibility;
advancing the guidewire through a blood vessel to a position adjacent the target site, wherein advancing the guidewire through a blood vessel includes disposing the hinge portion across a bifurcation in the blood vessel.

2. The method of claim 1, wherein advancing the guidewire through a blood vessel to a position adjacent the target site includes advancing the guidewire through a first artery and into a second artery through a junction between the first artery and a second artery, wherein the junction forming a transition region that defines a bend in the anatomy that creates an angle of sixty degrees or greater, and wherein the first artery is an abdominal aorta and the second artery is a renal artery, and the transition region is the junction between abdominal aorta and the renal artery.

3. The method of claim 1, wherein advancing the guidewire through a blood vessel to a position adjacent the target site includes advancing the guidewire through a first artery and into a second artery through a junction between the first artery and a second artery, wherein the junction forming a transition region that defines a bend in the anatomy that creates an angle of sixty degrees or greater, and wherein the first artery is a first femoral artery and the second artery is a second femoral artery, and the transition region is an aortic bifurcation.

4. The method of claim 1, wherein the slots formed in the proximal portion of the tubular member are disposed in a first density and wherein slots formed in the hinge portion are disposed in a second density different from the first density.

5. The method of claim 4, wherein the slots formed in the hinge portion are aligned on opposite sides of the tubular member such that the hinge portion has a greater amount of lateral flexibility in directions oriented toward the slots and a decreased amount of lateral flexibility in directions oriented orthogonal to the slots.

6. The method of claim 4, wherein the second density includes more slots per unity length than the first density.

7. The method of claim 4, wherein the slots formed in the distal portion of the tubular member are disposed in a third density.

8. The method of claim 7, wherein the third density is different from the first density, the second density, or both.

9. The method of claim 7, wherein the third density is the same as the first density.

10. The method of claim 1, wherein the guidewire includes a core wire disposed within the tubular member.

11. The method of claim 10, wherein the tubular member extends distally beyond a distal end of the core wire.

12. The method of claim 11, wherein a shaping member is attached to the distal end of the core wire.

13. The method of claim 1, wherein the proximal portion of the tubular member extends to a position adjacent to the proximal end of the guidewire.

14. A method for advancing a guidewire to a target site in the anatomy of a patient, the method comprising:
providing a guidewire, the guidewire including a tubular member having a plurality of slots formed therein;
wherein the tubular member has a proximal portion having slots disposed therein in a first density, a hinge portion having slots disposed therein in a second density different from the first density, and a distal portion having slots disposed therein in a third density, wherein the distal portion of the tubular member extends to a distal end of the guidewire;
advancing the guidewire through a blood vessel to a position adjacent the target site, wherein advancing the guidewire through a blood vessel includes disposing the hinge portion across a bifurcation in the blood vessel.

15. The method of claim 14, wherein the second density includes more slots per unit length than the first density.

16. The method of claim 15, wherein the second density includes more slots per unit length than both the first density and the third density.

17. The method of claim 14, wherein the third density is different from the first density, the second density, or both.

18. The method of claim 14, wherein the third density is the same as the first density.

19. The method of claim 14, wherein the proximal portion of the tubular member has a first outer diameter, the distal portion of the tubular member has a second outer diameter, and the hinge portion of the tubular member has a third outer diameter that is smaller than the first outer diameter, the second outer diameter, or both.

20. A hinged guidewire, comprising:
a tubular member having a plurality of slots formed therein;
wherein the tubular member has a proximal portion having slots disposed therein in a first density, a hinge portion having slots disposed therein in a second density different from the first density, and a distal portion having slots disposed therein in a third density that is the same as the first density;
wherein the second density includes more slots per unit length than both the first density and the third density; and
wherein the distal portion of the tubular member extends to a distal end of the guidewire.

21. The guidewire of claim 20, further comprising a core wire disposed within the tubular member.

22. A hinged guidewire, comprising:
a tubular member having a plurality of slots formed therein;
wherein the tubular member has a proximal portion having a plurality of slots formed therein, a hinge portion having a plurality of slots formed therein, and a distal portion having a plurality of slots formed therein;
wherein the slots in the hinge portion are aligned on opposite sides of the tubular member such that the hinge portion has a greater amount of lateral flexibility in directions oriented toward the slots and a decreased amount of lateral flexibility in directions oriented orthogonal to the slots; and
wherein the distal portion of the tubular member extends to a distal end of the guidewire.

23. The guidewire of claim 22, further comprising a core wire disposed within the tubular member.
